(19) United States
(12) Patent Application Publication
(10) Pub. No.: US 2026/0126378 A1
(43) Pub. Date: May 7, 2026

(54) MOISTURE CONTENT MEASUREMENT SYSTEM BASED ON MULTI-INFRARED DETECTION REFLECTION BANDS

(71) Applicant: ZHEJIANG UNIVERSITY, HANGZHOU (CN)

(72) Inventors: HAIBIN CUI, HANGZHOU (CN); FEI WANG, HANGZHOU (CN); GUOJUN LV, HANGZHOU (CN); QIYU GAO, HANGZHOU (CN); SONG LUO, HANGZHOU (CN)

(73) Assignee: ZHEJIANG UNIVERSITY, HANGZHOU (CN)

(21) Appl. No.: 19/365,231

(22) Filed: Oct. 22, 2025

(30) Foreign Application Priority Data
Nov. 1, 2024 (CN) 202411554330.5

(51) Int. Cl. G01N 21/3554; G01N 33/18

(52) U.S. Cl. CPC G01N 21/3554; G01N 33/18; G01N 2201/0686

(57) ABSTRACT

Provided is a moisture content measurement system based on multi-infrared detection reflection bands, including a bearing plate for carrying sludge; an infrared light source configured to emit infrared light for irradiating the sludge; an infrared detection array including several infrared detectors, wherein each infrared detector is equipped with an optical filter, which is configured to define a wavelength band range of infrared light received by the infrared detector, allowing only the infrared light within the wavelength band to pass through, and in turn enabling the infrared detector to output a corresponding voltage signal; and a signal processing module configured to receive and process the voltage signal output by the infrared detector. The moisture content measurement system enables rapid and non-contact measurement of sludge moisture content through infrared spectroscopy technology, without the need for sampling or damaging sludge samples, and meanwhile, is also conducive to realizing online real-time monitoring.

### CROSS-REFERENCE TO RELATED APPLICATIONS

This application claims priority of Chinese Patent Application No. 2024115543305, filed on Nov. 1, 2024, the contents of which are hereby incorporated by reference.

### TECHNICAL FIELD

The present application relates to the technical field of sludge moisture content measurement, and in particular, to a sludge moisture content measurement system based on multi-infrared detection reflection bands.

### BACKGROUND

In the sewage treatment process, the accurate measurement of sludge moisture content is crucial for optimizing the treatment process and improving treatment efficiency. Traditional sludge moisture content measurement methods mainly include the gravimetric method, drying method, etc. Although these methods have a certain degree of reliability, they often require taking sludge samples away from the site for processing, which is not only complex in operation and time-consuming, but also makes it difficult to achieve on-line real-time monitoring.

As a non-contact measurement technology, infrared spectroscopy technology has been widely used in moisture content measurement in recent years due to its characteristics of rapidity and non-contact. However, existing infrared spectroscopy measurement systems still have some limitations; especially in the measurement of sludge with complex components, when infrared detectors with a single wavelength band face interference from multiple components, it is difficult to effectively distinguish the absorption signals associated with moisture in sludge and the absorption signals associated with other components, which leads to a decrease in the accuracy and stability of measurement.

### SUMMARY

### (I) Technical Problems to be Solved

In view of the shortcomings in the prior art, the present application provides a sludge moisture content measurement system based on multi-infrared detection reflection bands, which has the advantage of being able to collect signals from a characteristic band and a reference band respectively through a plurality of infrared detectors, and combine model construction and data processing to accurately measure a sludge moisture content.

### (II) Technical Solutions

To achieve the above-mentioned objective of being able to collect signals from a characteristic band and a reference band respectively through a plurality of infrared detectors, and combine model construction and data processing to accurately measure a sludge moisture content, the present application provides the following technical solution: a sludge moisture content measurement system based on multi-infrared detection reflection bands includes a bearing plate for carrying sludge, and further includes:
an infrared light source configured to emit infrared light for irradiating the sludge;
an infrared detection array including several infrared detectors, where each infrared detector is equipped with an optical filter, and the optical filter is configured to define a wavelength band range of infrared light received by the infrared detector, allowing only the infrared light within the wavelength band to pass through, and in turn enabling the infrared detector to output a corresponding voltage signal;
a signal processing module configured to receive and process the voltage signal output by the infrared detector;
a model construction module configured to construct a correlation model between reflected light power and moisture content; and
a moisture content calculation module configured to calculate a sludge moisture content based on the correlation model.

As a preferred technical solution of the present application, the signal processing module includes:
a signal amplification and filtering unit configured to receive and process the voltage signal output by the infrared detector, amplifying the signal and eliminating noise interference;
an analog-to-digital conversion unit configured to convert an analog signal into a high-precision digital signal; and
a data storage unit configured to store the processed voltage signal.

As a preferred technical solution of the present application, the infrared detectors include:
a characteristic band detector, where the optical filter of the characteristic band detector is configured to receive infrared light affected by absorption and scattering by sludge moisture, and output a characteristic voltage signal; and
a reference band detector, where the optical filter of the reference band detector is configured to receive infrared light not affected by absorption and scattering by sludge moisture, and output a reference voltage signal;
where the model construction module constructs the correlation model between the reflected light power and the moisture content based on the characteristic voltage signal from the characteristic band detector and the reference voltage signal from the reference band detector.

As a preferred technical solution of the present application, the sludge moisture content measurement system based on multi-infrared detection reflection bands further includes:
a water vapor measurement channel including a temperature sensor and a humidity sensor configured to measure temperature and relative humidity in the air and calculate an absolute humidity;
where an interference of absorption by water vapor in the air on an infrared reflection signal is corrected through a value of the absolute humidity.

As a preferred technical solution of the present application, a correction process includes:
determining a coefficient of absorption by the water vapor in a specific infrared wavelength band according to the value of the absolute humidity of the water vapor, compensating for a voltage attenuation amount caused by the absorption by the water vapor in combination with a reference voltage signal from the reference band detector, and obtaining a corrected voltage signal after eliminating the interference by the water vapor;

As a preferred technical solution of the present application, the sludge moisture content measurement system based on multi-infrared detection reflection bands further includes a focusing lens disposed below the optical filter.

As a preferred technical solution of the present application, the sludge moisture content measurement system based on multi-infrared detection reflection bands further includes a fixed plate with an inner cavity formed therein, where a top cylinder communicating with the inner cavity is fixedly installed on an edge side of a top portion of the fixed plate, and a bottom cylinder communicating with the inner cavity is fixedly installed on an edge side of a bottom portion of the fixed plate; and the infrared detector is mounted in the top cylinder, and the focusing lens is fixed in the bottom cylinder;
where a switching disk rotatably installed in the inner cavity, several mounting openings for installing optical filters are uniformly formed in the switching disk.

As a preferred technical solution of the present application, a sensor fixing housing is fixedly installed on an outer wall of the bottom cylinder, and a measurement sensor is fixed in the sensor fixing housing.

As a preferred technical solution of the present application, the sludge moisture content measurement system based on multi-infrared detection reflection bands further includes an exhaust pipe with an air jet head disposed at its end;
where the air jet head at the end of the exhaust pipe is inserted into the bottom cylinder and faces the focusing lens.

### (III) Beneficial Effects

Compared with the prior art, the present application provides a sludge moisture content measurement system based on multi-infrared detection reflection bands, which has the following beneficial effects:
1. The sludge moisture content measurement system based on multi-infrared detection reflection bands can simultaneously acquire infrared light information of multiple wavelengths by using a plurality of infrared detectors equipped with filters, thereby improving the accuracy and stability of moisture content measurement.
2. The sludge moisture content measurement system based on multi-infrared detection reflection bands adds the water vapor measurement channel, acquires the absolute humidity of the water vapor, and uses the absolute humidity to correct an infrared light signal, eliminates the interference caused by the absorption by the water vapor, and can obtain more accurate moisture content measurement results.
3. The sludge moisture content measurement system based on multi-infrared detection reflection bands enables rapid and non-contact measurement of sludge moisture content through infrared spectroscopy technology, without the need for sampling or damaging sludge samples, and meanwhile, is also conducive to realizing online real-time monitoring.

### BRIEF DESCRIPTION OF THE DRAWINGS

FIG. 1 is a schematic diagram of a measurement system of the present application;

FIG. 2 is an enlarged schematic diagram of a fixed plate part of the present application;

FIG. 3 is a cross-sectional view of the fixed plate part of the present application;

FIG. 4 is an enlarged schematic diagram of a switching disk part of the present application;

FIG. 5 is a cross-sectional view of a fixed plate part in another embodiment of the present application; and

FIG. 6 is an enlarged schematic diagram of a piston cylinder part of the present application.

In the figures: 1. Bearing plate; 2. Infrared light source; 3. Light source support; 4. Infrared detector; 5. Optical filter; 6. Focusing lens; 7. Fixed plate; 8. Inner cavity; 9. Switching disk; 10. Mounting opening; 11. Top cylinder; 12. Bottom cylinder; 13. Sensor fixing housing; 14. Measurement sensor; 15. Vertical shaft; 16. Fixed bearing; 17. Driving disk; 18. Arc-shaped groove; 19. Column groove; 20. Driving shaft; 21. Positioning disk; 22. Eccentric disk; 23. Sliding column; 24. Pressing plate; 25. Connecting rod; 26. Piston; 27. Piston cylinder; 28. Support spring; 29. Intake pipe; 30. Exhaust pipe.

### DETAILED DESCRIPTION OF THE EMBODIMENTS

The technical solutions in the embodiments of the present application will be clearly and completely described below in conjunction with the accompanying drawings in the embodiments of the present application. Apparently, the described embodiments are only a part of the embodiments of the present application, rather than all the embodiments. Based on the embodiments of the present application, all other embodiments obtained by those of ordinary skill in the art without creative effects shall fall within the protection scope of the present application.

### Embodiment One

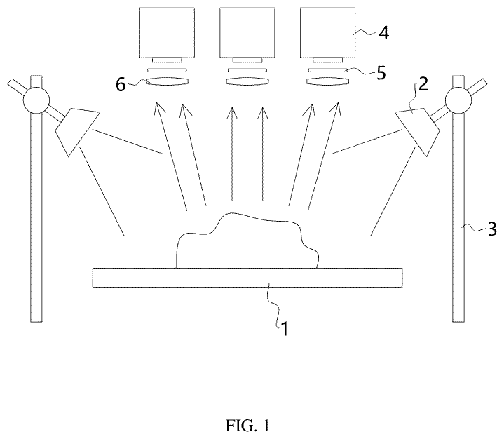

Referring to FIG. 1, a sludge moisture content measurement system based on multi-infrared detection reflection bands includes a bearing plate 1 for carrying sludge. As shown in FIG. 1, an infrared light source 2 is further provided, which is configured to emit infrared light for irradiating the sludge.

An infrared detection array is disposed above the sludge, the infrared detection array includes several infrared detectors 4, each infrared detector 4 is equipped with an optical filter 5, and the optical filter 5 is configured to define a wavelength band range of infrared light received by the infrared detector 4, allowing only the infrared light within the wavelength band to pass through, and in turn enabling the infrared detector 4 to output a corresponding voltage signal; and then, the voltage signal output by the infrared detector 4 is received and processed by a signal processing module.

In this embodiment, the signal processing module includes a signal amplification and filtering unit, an analog-to-digital conversion unit, and a data storage unit. The signal amplification and filtering unit is configured to receive and process the voltage signal output by the infrared detector 4, amplifying the signal and eliminating noise interference; the analog-to-digital conversion unit is configured to convert an analog signal into a high-precision digital signal; and the data storage unit is configured to store the processed voltage signal.

In the present application, the infrared detectors 4 are specifically divided into a characteristic band detector and a reference band detector. The optical filter 5 of the characteristic band detector is configured to receive infrared light affected by absorption and scattering by sludge moisture, and output a characteristic voltage signal (for example, an optical filter 5 with a central wavelength of 1400 nm and a bandwidth of 24 nm is selected). The optical filter 5 of the reference band detector is configured to receive infrared light not affected by absorption and scattering by sludge moisture, and output a reference voltage signal (for example, an optical filter 5 with a central wavelength of 1100 nm and a bandwidth of 20 nm is selected). The specific selection of the optical filter 5 can be made by those of skill in the art according to measurement requirements.

A model construction module constructs a correlation model between reflected light power and moisture content based on the characteristic voltage signal from the characteristic band detector and the reference voltage signal from the reference band detector.

For the construction of the correlation model, first, the voltage signals of reflected light are collected through the infrared detectors 4, and the voltage signals V_{f} and Vᵣ for the characteristic band and the reference band, respectively, are recorded. The voltage signal for the characteristic band is affected by the moisture content of the sludge, while the voltage signal for the reference band is used for error correction.

To eliminate the influence of environmental noise and other external interferences, the voltage signals for the characteristic band and the reference band are subjected to differential processing, so as to obtain a relative variation ΔV:

$$Δ V = {V}_{r} - {V}_{f}$$

In the present application, a quadratic polynomial model is used to fit a relationship between the reflected light power and the moisture content, and the sludge moisture content W and a voltage variation ΔV can be expressed as:

$$W = a \cdot {( Δ V )}^{2} + b \cdot ( Δ V ) + c ,$$

where a, b, and c are to-be-fitted model parameters, and W is an actual moisture content of the sludge.

By collecting a large amount of data and using the method of regression analysis, the parameters a, b, and c can be fitted; and taking the actual moisture content W of each sample and a corresponding voltage variation ΔV as input data, the least squares method is used to fit the model parameters:

$$min {{\sum }_{i = 1}^{n} [ W i - ( a \cdot {( Δ V i )}^{2} + b \cdot ( Δ V i ) + c ) ]}^{2} .$$

Through the above optimization, the values of a, b, and c can be obtained, and the fitting error of the model is relatively small.

After the construction of the model is completed, the model can be used for calculating the sludge moisture content. In calculation, ΔV is substituted into the fitted model, the sludge moisture content can be obtained (i.e., a moisture content calculation module).

In the present application, a water vapor measurement channel is further provided, which specifically includes a temperature sensor and a humidity sensor configured to measure temperature and relative humidity in the air and calculate an absolute humidity. An interference of absorption by water vapor in the air on an infrared reflection signal can be corrected through a value of the absolute humidity.

A specific correction method is: determining a coefficient of absorption by the water vapor in a specific infrared wavelength band according to the value of the absolute humidity of the water vapor, compensating for a voltage attenuation amount caused by the absorption by the water vapor in combination with a reference voltage signal from the reference band detector, and obtaining a corrected voltage signal after eliminating the interference by the water vapor; the corrected voltage signal is used for constructing the correlation model, thereby being capable of ensuring that a final reflected signal only includes the light intensity attenuation caused by the absorption by the moisture on a surface of the sludge, and in turn improving the measurement accuracy of the sludge moisture content.

### Embodiment Two

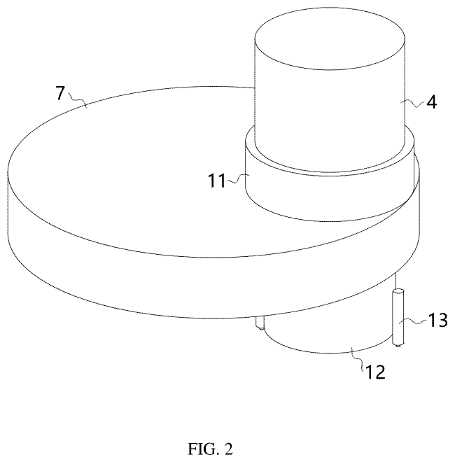

Referring to FIGS. 1-4, on the basis of Embodiment One, the infrared light source 2 in this embodiment may be a continuous-spectrum infrared lamp or a laser with a specific wavelength. The infrared light source 2 is installed on an adjustable light source support (3), and parameters such as height and angle can be adjusted.

As shown in FIG. 1, there is further provided a focusing lens 6, which is disposed below the optical filter 5 and functions like a magnifying glass, increasing the light intensity entering the effective detection area of the infrared detector 4.

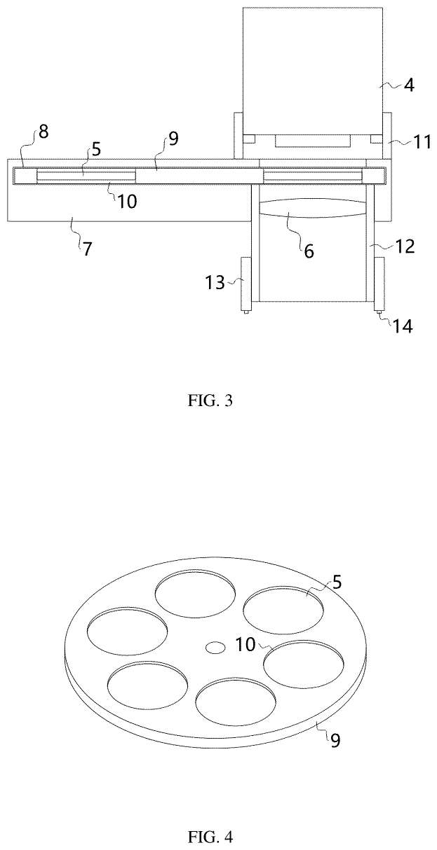

In the present application, as shown in FIG. 3, there is further provided a fixed plate 7 with an inner cavity 8 formed therein; a top cylinder 11 communicating with the inner cavity 8 is fixedly installed on an edge side of a top portion of the fixed plate 7, and a bottom cylinder 12 communicating with the inner cavity 8 is fixedly installed on an edge side of a bottom portion of the fixed plate 7; the infrared detector 4 is mounted in the top cylinder 11, and the focusing lens 6 is fixed in the bottom cylinder 12; and a switching disk 9 is rotatably installed in the inner cavity 8, several mounting openings 10 for installing optical filters 5 are uniformly formed in the switching disk 9, and the optical filters 5 are installed in the mounting openings 10, respectively.

By rotating the switching disk 9, the optical filters 5 at different positions on the switching disk 9 can be aligned with the infrared detector 4 and the focusing lens 6, thereby realizing convenient switching of the optical filters 5.

As shown in FIG. 3, a sensor fixing housing 13 is fixedly installed on an outer wall of the bottom cylinder 12, and a measurement sensor 14 is fixed in the sensor fixing housing 13. The measurement sensor 14 includes a temperature sensor and a humidity sensor configured to measure temperature and relative humidity in the air and calculate an absolute humidity; and in turn, an interference of absorption by water vapor in the air on an infrared reflection signal can be corrected through a value of the absolute humidity.

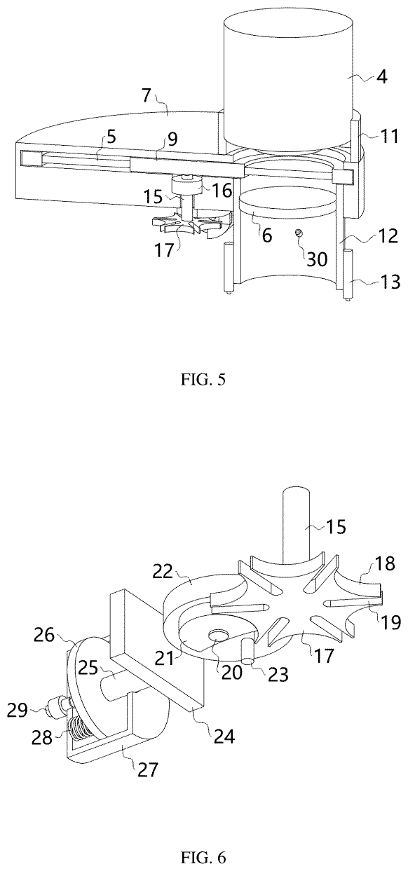

As shown in FIG. 5, there is further provided an exhaust pipe 30 with a jet head connected to its end; the jet head at the end of the exhaust pipe 30 is inserted into the bottom cylinder 12 and faces the focusing lens 6, and the gas ejected therefrom is sprayed on the bottom of the focusing lens 6, thereby being capable of realizing effective cleaning of the focusing lens 6; and since the optical filter 5 is always located in the inner cavity 8 and the infrared detector 4 is fixed in the top cylinder 11 without contacting the external environment, no cleaning is required.

### Embodiment Three

Referring to FIGS. 5 and 6, on the basis of Embodiment One and Embodiment Two, this embodiment specifically provides a mechanism for driving the switching disk 9 to rotate, as shown in FIG. 5.

A vertical shaft 15 is fixedly installed at the center of a bottom portion of the switching disk 9; the vertical shaft 15 is fixed through a fixed bearing 16, and the bottom end of the vertical shaft 15 is fixedly connected to a driving disk 17; and column grooves 19 are uniformly formed on the driving disk 17, and arc-shaped grooves 18 are formed by recessing between adjacent column grooves 19.

In addition, there is provided a driving shaft 20 driven by a motor (not shown in the figure); a positioning disk 21 and an eccentric disk 22 are fixedly installed on the driving shaft 20; the positioning disk 21 corresponds to the arc-shaped grooves 18; and a sliding column 23 is fixedly installed on the eccentric disk 22, and the sliding column 23 can slide into or out of the column grooves 19.

When the motor drives the driving shaft 20 to rotate, the driving shaft 20 also drives the positioning disk 21 and the eccentric disk 22 to rotate synchronously. When the eccentric disk 22 rotates, the sliding column 23 thereon can slide into one column groove 19, then drive the driving disk 17 to rotate by a certain angle, and then slide out of the column groove 19, realizing the rotation control of the driving disk 17 at a directional angle, and in turn realizing the switching of different optical filters 5.

The positioning disk 21 serves to position the driving disk 17; meanwhile, the positioning disk 21 is also provided with a groove, which is used to allow the “sharp corners” of the driving disk 17 to pass through it.

In the present application, as shown in FIG. 6, a piston cylinder 27 is further fixedly installed at the bottom portion of the fixed plate 7; a piston 26 is movably disposed in the piston cylinder 27; one side of the piston 26 is supported by a support spring 28, and the other side is fixedly connected to a pressing plate 24 through a connecting rod 25; the pressing plate 24 is arranged close to the eccentric disk 22, and when the eccentric disk 22 rotates, it can push the pressing plate 24 cyclically.

In this embodiment, an intake pipe 29 is connected through to the piston cylinder 27, and the intake pipe 29 is provided with a one-way intake valve; an exhaust pipe 30 is also connected through to the piston cylinder 27, and the exhaust pipe 30 is provided with a one-way exhaust valve.

When the eccentric disk 22 rotates to push the pressing plate 24 cyclically, the movement of the piston 26 can be utilized to pump the air sucked in from the intake pipe 29 out from the jet head at the end of the exhaust pipe 30, thereby realizing effective cleaning of the focusing lens 6 without an additional gas generating device or an additional electronic control system.

Although the embodiments of the present application have been shown and described, for those of ordinary skill in the art, it can be understood that various changes, modifications, substitutions and variations can be made to these embodiments without departing from the principle and spirit of the present application. The scope of the present application is defined by the appended claims and their equivalents.

What is claimed is:

1. A sludge moisture content measurement system based on multi-infrared detection reflection bands, comprising a bearing plate (1) for carrying sludge, and further comprising:
an infrared light source (2) configured to emit infrared light for irradiating the sludge;
an infrared detection array comprising several infrared detectors (4), wherein each infrared detector (4) is equipped with an optical filter (5), and the optical filter (5) is configured to define a wavelength band range of infrared light received by the infrared detector (4), allowing only the infrared light within the wavelength band to pass through, and in turn enabling the infrared detector (4) to output a corresponding voltage signal;
a signal processing module configured to receive and process the voltage signal output by the infrared detector (4);
a model construction module configured to construct a correlation model between reflected light power and moisture content; and
a moisture content calculation module configured to calculate a sludge moisture content based on the correlation model;
wherein a quadratic polynomial model is used to fit a relationship between the reflected light power and the moisture content, and the sludge moisture content W and a voltage variation ΔV can be expressed as: W=a·(ΔV)²+b·(ΔV)+c, where a, b, and c are to-be-fitted model parameters, and W is an actual moisture content of the sludge;
the sludge moisture content measurement system based on multi-infrared detection reflection bands further comprises:
a water vapor measurement channel comprising a temperature sensor and a humidity sensor configured to measure temperature and relative humidity in the air and calculate an absolute humidity; wherein
an interference of absorption by water vapor in the air on an infrared reflection signal is corrected through a value of the absolute humidity; and
a correction process comprises: determining a coefficient of absorption by the water vapor in a specific infrared wavelength band according to the value of the absolute humidity of the water vapor, compensating for a voltage attenuation amount caused by the absorption by the water vapor in combination with a reference voltage signal from a reference band detector, and obtaining a corrected voltage signal after eliminating the interference by the water vapor;
further comprising a focusing lens (6) disposed below the optical filter (5);
further comprising a fixed plate (7) with an inner cavity (8) formed therein, wherein a top cylinder (11) communicating with the inner cavity (8) is fixedly installed on an edge side of a top portion of the fixed plate (7), and a bottom cylinder (12) communicating with the inner cavity (8) is fixedly installed on an edge side of a bottom portion of the fixed plate (7); and the infrared detector (4) is mounted in the top cylinder (11), and the focusing lens (6) is fixed in the bottom cylinder (12);
a switching disk (9) is rotatably installed in the inner cavity (8), several mounting openings (10) for installing optical filters (5) are uniformly formed in the switching disk (9);
a sensor fixing housing (13) is fixedly installed on an outer wall of the bottom cylinder (12), and a measurement sensor (14) is fixed in the sensor fixing housing (13); and
further comprising an exhaust pipe (30) with an air jet head disposed at its end;
wherein the air jet head at the end of the exhaust pipe (30) is inserted into the bottom cylinder (12) and faces the focusing lens (6).

2. The sludge moisture content measurement system based on multi-infrared detection reflection bands according to claim 1, wherein the signal processing module comprises:
a signal amplification and filtering unit configured to receive and process the voltage signal output by the infrared detector (4), amplifying the signal and eliminating noise interference;
an analog-to-digital conversion unit configured to convert an analog signal into a high-precision digital signal; and
a data storage unit configured to store the processed voltage signal.

3. The sludge moisture content measurement system based on multi-infrared detection reflection bands according to claim 1, wherein the infrared detectors (4) comprise:
a characteristic band detector, wherein the optical filter (5) of the characteristic band detector is configured to receive infrared light affected by absorption and scattering by sludge moisture, and output a characteristic voltage signal; and
the reference band detector, wherein the optical filter (5) of the reference band detector is configured to receive infrared light not affected by absorption and scattering by sludge moisture, and output a reference voltage signal;
wherein the model construction module constructs the correlation model between the reflected light power and the moisture content based on the characteristic voltage signal from the characteristic band detector and the reference voltage signal from the reference band detector.

4. The sludge moisture content measurement system based on multi-infrared detection reflection bands according to claim 2, wherein the infrared detectors (4) comprise:
a characteristic band detector, wherein the optical filter (5) of the characteristic band detector is configured to receive infrared light affected by absorption and scattering by sludge moisture, and output a characteristic voltage signal; and
the reference band detector, wherein the optical filter (5) of the reference band detector is configured to receive infrared light not affected by absorption and scattering by sludge moisture, and output a reference voltage signal;
wherein the model construction module constructs the correlation model between the reflected light power and the moisture content based on the characteristic voltage signal from the characteristic band detector and the reference voltage signal from the reference band detector.
